(19) United States
(12) Patent Application Publication
(10) Pub. No.: US 2014/0031715 A1
(43) Pub. Date: Jan. 30, 2014

(54) COIL ELECTRODE APPARATUS FOR THERMAL THERAPY FOR TREATING BONE TISSUE

(71) Applicants: Michael David SHERAR, Toronto (CA); Kieran MURPHY, Toronto (CA); Brock MILLER, Mahwah, NJ (US); Claire MCCANN, Toronto (CA); Cari Marisa WHYNE, Toronto (CA); Padina Sadat PEZESHKI, Toronto (CA); Albert Juang Ming YEE, Toronto (CA)

(72) Inventors: Michael David SHERAR, Toronto (CA); Kieran MURPHY, Toronto (CA); Brock MILLER, Mahwah, NJ (US); Claire MCCANN, Toronto (CA); Cari Marisa WHYNE, Toronto (CA); Padina Sadat PEZESHKI, Toronto (CA); Albert Juang Ming YEE, Toronto (CA)

(21) Appl. No.: 13/954,647

(22) Filed: Jul. 30, 2013

Related U.S. Application Data
(60) Provisional application No. 61/677,367, filed on Jul. 30, 2012.

(51) Int. Cl. A61B 18/18

(52) U.S. Cl. CPC A61B 18/18
USPC 600/562; 607/101

(57) ABSTRACT

Various embodiments are described herein for a coil electrode, RF applicator and RF treatment apparatus, along with an associated method of operation, that have been developed for treating tumors in bone tissue, including large tumors with a single treatment session or ablating intravertebral nerves. Embodiments are also described that provide the capability to perform multifocal treatments for safe and effective treatment of multiple bone tumors or ablating intravertebral nerves. Embodiments are also described that provide a 3-tiered multi-modality treatment regimen which would sequentially include RF ablation of a bone tumor, tumor debulking and vertebroplasty or ablating intravertebral nerves as part of pain treatment.

### CROSS REFERENCED TO RELATED APPLICATION

This application claims the benefit of U.S. Provisional Application No. 61/677,367 filed on Jul. 30, 2012 and the contents of Application No. 61/677,367 are hereby incorporated by reference in their entirely.

### FIELD

Various embodiments are described herein that relate to an apparatus and method for using an RF electrode to treat bone tissue.

### BACKGROUND

Radiofrequency ablation (RFA) is a rapidly expanding new technology for the treatment of cancer. Minimally invasive technologies are revolutionizing primary cancer treatment and in many cases gradually replacing surgery. Conventional RFA technology provides thermal destruction by exciting ions in a tissue and their respective agitations heat the exposed region. The thermal destruction causes coagulative necrosis with a target temperature greater than 55° C. Three groups of patients benefit from these advances. Those who would normally undergo surgery benefit because the minimally invasive technology results in less trauma, lower complication rates and shorter or no hospital stays. A second group includes those patients who may be too ill for surgery or whose tumors are in close proximity to critical normal tissues such that surgery cannot be performed. A third group includes those patients who may either have multiple local metastases that need treatment or whose tumors may be too large for surgical intervention.

### SUMMARY

A coil electrode, along with an associated method of operation, has been developed for use in an RF applicator, RFA apparatus or RFA system for heating tumors, including large tumors with a single heating session. The coil electrode generally has a helical geometry, although many variations exist, and is provided with an excitation current having a frequency that is sufficient for magnetic induction and coupling of various electric and magnetic fields to produce an electric field within the volume surrounded by the coil for directly applying heat to the tissue or material therein.

In another embodiment, this device can be used in a 3-tiered multi-modality treatment regimen, which can sequentially include RF ablation of tissue, tumor debulking and vertebroplasty.

In another embodiment, this device can be used in a 2-tiered regimen, including RF ablation followed by vertebroplasty (without debulking). In RF ablation of tissue, there is also the capability to perform multifocal treatments for safe and effective treatment of multiple bone tumors. Other variations are also possible as described herein.

Accordingly, in one aspect, at least one embodiment described herein provides an apparatus for providing RF treatment. The apparatus comprises an RF applicator comprising an applicator housing; an applicator cannula mounted at a distal end of the applicator housing, the applicator cannula including a tip spaced apart from a proximal end of the applicator housing; an electrode disposed within the applicator cannula and the applicator housing, the electrode having a retracted position within the applicator cannula and a deployed position outside of the tip of the applicator cannula; an actuator mounted at the proximal end of the applicator housing; a mechanical assembly disposed within the applicator housing to mechanically couple the actuator and the electrode, the mechanical assembly being configured to convert a rotational movement of the actuator to a longitudinal movement of the electrode with respect to the applicator cannula; and an electrical connection assembly configured to electrically couple the electrode to a power source.

In another aspect, at least one embodiment described herein provides a method for providing RF treatment to bone or an intravertebral nerve. The method comprises forming a guidance hole adjacent to a target site in the bone; forming a toroidal void around the target site using a first coil having a first diameter and then retracting the first coil; deploying a coil electrode having a second diameter in the toroidal void, the second diameter being smaller than the first diameter; and applying RF energy to the coil electrode to ablate the target site.

In another aspect, at least one embodiment described herein provides a method for providing bilateral RF treatment to bone or an intravertebral nerve. The method comprises forming a first guidance hole adjacent to a first target site in a vertebrae; forming a second guidance hole adjacent to a second target site in the vertebra, the second target site being located laterally opposite the first target site within the vertebra; inserting and then deploying a first coil electrode at the first target site; inserting and then deploying a second coil electrode at the second target site; and applying RF energy to the first and second coil electrodes to ablate the first and second target sites.

In another aspect, at least one embodiment described herein provides a method for providing multifocal RF treatment to bone or an intravertebral nerve. The method comprises forming a first guidance hole adjacent to a first target site in a first vertebra; forming a second guidance hole adjacent to a second target site in a second vertebra; inserting and then deploying a first coil electrode at the first target site; inserting and then deploying a second coil electrode at the second target site; and applying an excitation signal to the first and second coil electrodes.

Other features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

For a better understanding of the various embodiments described herein and to show more clearly how these various embodiments may be carried into effect, reference will now be made, by way of example only, to the accompanying drawings which show at least one example embodiment and in which:

FIG. 1 shows an example embodiment of an RF treatment apparatus;

FIGS. 2A-2B show an example of an alternative embodiment of an RF applicator;

FIGS. 3A-3F show an example use of an RF applicator within the 3-tier multi-modality regimen described above;

FIG. 4 shows an example of a jig that can be used for setting the shape of the helical portion of a coil electrode during annealing;

FIG. 5 shows a portion of an example RF applicator with a loosely wound coil and guidance groove on the cannula;

FIG. 6 shows a portion of an example RF applicator with a tightly wound coil and a guidance groove on the cannula;

FIGS. 7A-7B show a top and side view, respectively, of an example of a lateral transpedicular coil placement;

FIG. 8 shows an example of a bilateral transpedicular coil placement;

FIGS. 9A-9B show an example of a multifocal treatment setup;

FIG. 10 shows a partial cross-sectional view of the RF applicator;

FIG. 11 shows an example of a bone coil electrode that was used in an ex-vivo test in porcine liver tissue;

FIG. 12 shows a cable for use with an RF treatment apparatus;

FIGS. 13A-13B show an example coil electrode in excised (i.e. ex vivo) healthy porcine lumbar vertebrae under CT fluoroscopic guidance;

FIGS. 14A-14B show an example coil electrode in an excised porcine lumbar vertebrae tumor;

FIG. 15A-15B show an example coil electrode in excised healthy human cadaveric lumbar vertebrae;

FIG. 16 is a block diagram of an example embodiment of an RF treatment apparatus;

FIG. 17 is block diagram of an example embodiment of an RF applicator;

FIG. 18 is a flowchart of an example embodiment of an RF bone tissue treatment method;

FIG. 19 is a flowchart of an example embodiment of an RF bone tissue bilateral method treatment; and

FIG. 20 is a flowchart of an example embodiment of an RF bone tissue multi-modal treatment method.

### DETAILED DESCRIPTION

Various devices or processes will be described below to provide examples of at least one embodiment of the claimed subject matter. No embodiment described below limits any claimed subject matter and any claimed subject matter may cover processes or devices that differ from those described below. The claimed subject matter is not limited to devices or processes having all of the features of any one device or process described below or to features common to multiple or all of the devices or processes described below. It is possible that a device or process described below is not an embodiment of any claimed subject matter. Any subject matter disclosed in a device or process described below that is not claimed in this document may be the subject matter of another protective instrument, for example, a continuing patent application, and the applicant, inventor or owners do not intend to abandon, disclaim or dedicate to the public any such subject matter by its disclosure in this document.

Furthermore, it will be appreciated that numerous specific details are set forth in order to provide a thorough understanding of the various embodiments described herein. However, it will be understood by those of ordinary skill in the art that the various embodiments may be implemented without these specific details. In other instances, well-known methods, procedures and components have not been described in detail so as not to obscure the embodiments described herein. Further, where considered appropriate, reference numerals may be repeated among the figures to indicate corresponding or analogous elements.

It should also be noted that the term coupled as used herein can have several different meanings depending in the context in which the term is used. For example, the term coupling can have a mechanical or electrical, connotation. For example, in some contexts, the term coupling indicates that two elements or devices can be directly physically connected to one another or connected to one another through one or more intermediate elements or devices via a physical coupling, such as a wire or cable, for example, or an electrical coupling, such as an electric or magnetic signal, for example.

It should be noted that terms of degree such as “substantially”, “about” and “approximately” as used herein mean a reasonable amount of deviation of the modified term such that the end result is not significantly changed. These terms of degree should be construed as including a certain deviation of the modified term if this deviation would not negate the meaning of the term that it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means up to a certain plus or minus change of the number to which reference is being made if this deviation would not negate the meaning of the term that it modifies.

Furthermore, in the following passages, different aspects of the embodiments are defined in more detail. Each aspect so defined may be combined with any other aspect or aspects unless clearly indicated to the contrary. In particular, any feature indicated as being preferred or advantageous may be combined with at least one other feature or features indicated as being preferred or advantageous.

Radiofrequency ablation (RFA) has been receiving increasing attention for the treatment of bone disease including spinal metastases, osteoid osteomas and chondroblastomas [4, 5]. Spinal metastastic disease is the most common tumor of the spine. Each year, about 5% of cancer patients develop spinal metastases from primary sites including breast, lung and prostate [4]. This causes progressive bone destruction that may result in debilitating pain, fractures and cord compression. These patients have a median survival of 10 months, and palliation of symptoms is the primary clinical objective. Skeletal metastases is also a common problem occurring in up to 85% of patients with the three most common types of cancer (i.e. breast, prostate, and lung cancer), at the time of death.

The current standard of care combines steroids, radiotherapy and surgery. Radiotherapy is the standard of care in patients who are surgically inoperable or have a poor survival prognosis. Radiotherapy is effective in reducing tumor size, and reducing pain and/or neurologic impairment. However radiotherapy as a stand-alone treatment for spinal tumors cannot correct biomechanical abnormalities, stabilize the spine, nor can it prevent vertebral body collapse. Open surgical procedures including decompression, and stabilization for indications such as spinal instability, cord compression, radio-resistant disease, previous exposure of the spinal cord to radiation, and/or debilitating pain may be an option for patients who are medically and surgically able with a life expectancy of at least 3 months, but have high rates of surgical morbidity and mortality [6]. In addition, multifocal vertebral lesions are common and may not be amenable to surgical treatment [7].

Percutaneous image guided vertebral body augmentation, such as vertebroplasty, is a less invasive surgical procedure for treating patients with spinal metastasis [8]. In this case, polymethylmethacrylate cement (PMMA) is injected into the tumor-affected vertebral body under image guidance. However, injection of bone cement into a vertebral body with a resident tumor can potentially cause tumor cell extravasation and venos embolization [5].

RFA, either alone or in combination with vertebroplasty, has been shown to be feasible in the treatment of pain from spinal tumors [5]. However, one of the major challenges of using current RFA technologies for the treatment of tumors in vertebral bodies as well as soft tissue targets is that complete tumor cell kill may not be achieved because power deposition and subsequent heating is limited to the vicinity immediately surrounding the radiofrequency electrode tines or needles, particularly in highly vascular targets. A further major challenge of using RFA for spinal tumors in particular, is the difficulty associated with protecting the spinal cord and nerves from damage if heating is deposited very close to the posterior vertebral body wall or in epidural tissue, while still producing a lesion of sufficient size to completely ablate the tumor target. Thus, challenges such as lesion size, conformality and non-uniform heating, which can limit the therapeutic efficacy of traditional RFA in large soft tissue targets, persist in the context of spinal tumors. In addition, difficulties associated with reliable and reproducible deployment using current expandable RFA array technologies may be even more of an issue in spinal tumors, in which a heterogeneous mix of diseased tissue as well as hard and soft bone may be present.

Disc related back pain is transmitted by nerves which pass through the end plates into the disc and leave the vertebral body either through the autonomic or somatic nervous system. This complex innervation includes transmission anteriorally of pain fibres through the sympathetic system and posteriorally through the sinuvertebral nerve. Back pain of vertebrogentic origin is also transmitted in this fashion and is very difficult to treat. RF energy can be used to destroy these nerves and denervate the vertebra and discs. This treatment can be done centrally or bilaterally and may need to be done above and below the disc space of interest.

RFA is also used as a pain relief technique for those with back pain and multiple other pain syndromes. When the lesion encompasses a painful nerve, the pain signals are interrupted and pain perception by the brain is reduced. In a recent clinical research study for patients treated with radiofrequency therapy, 21% had complete pain relief and 65% reported mild to moderate pain relief. The majority of the respondents reported reduction in the use of pain medications [9].

Ablation of the sinuvertebral nerve can be achieved with the RFA device and the associated methods described herein. When performed at multiple levels, sinuvertebral nerve ablation can be performed as part of a spinal fusion procedure or as part of a stand-alone pain spine intervention. For example, in some embodiments, intravertebral nerve ablation can employ the three or two-tiered multi-modality approaches described in accordance with the teachings herein. In some embodiments, intravertebral nerve ablation can also employ the bilateral treatment method described in accordance with the teachings herein. In some embodiments, intravertebral nerve ablation can also employ the multifocal treatment method described in accordance with the teachings herein.

The embodiments described herein generally relate to an apparatus and method for heating a target bone tissue region. An electrode and an RF applicator, along with associated methods of operation, have been developed for use in an RF apparatus for treating bone tumors or intravertebral nerves by heating these elements, even larger tumors, with a single treatment session in at least some cases.

In at least one embodiment described herein, when a coil electrode having a solenoidal geometry is supplied with energy at frequencies sufficient for magnetic induction, the coil electrode produces uniform electric fields throughout its center volume leading to uniform heating. An applicator that uses such a coil electrode can treat tumors of variable dimensions by changing the diameter and pitch of the coil. Such an applicator combines a robust geometry with uniform and predictable heating patterns as well as a robust deployment mechanism and methodology to deploy the electrode into bone tissue in order to heat various types of bone tumors, such as large spinal neoplasms for example, while still ensuring critical structures such as the spinal cord are spared. Preliminary testing has demonstrated the potential of this device for the treatment of tumors in vertebral bodies.

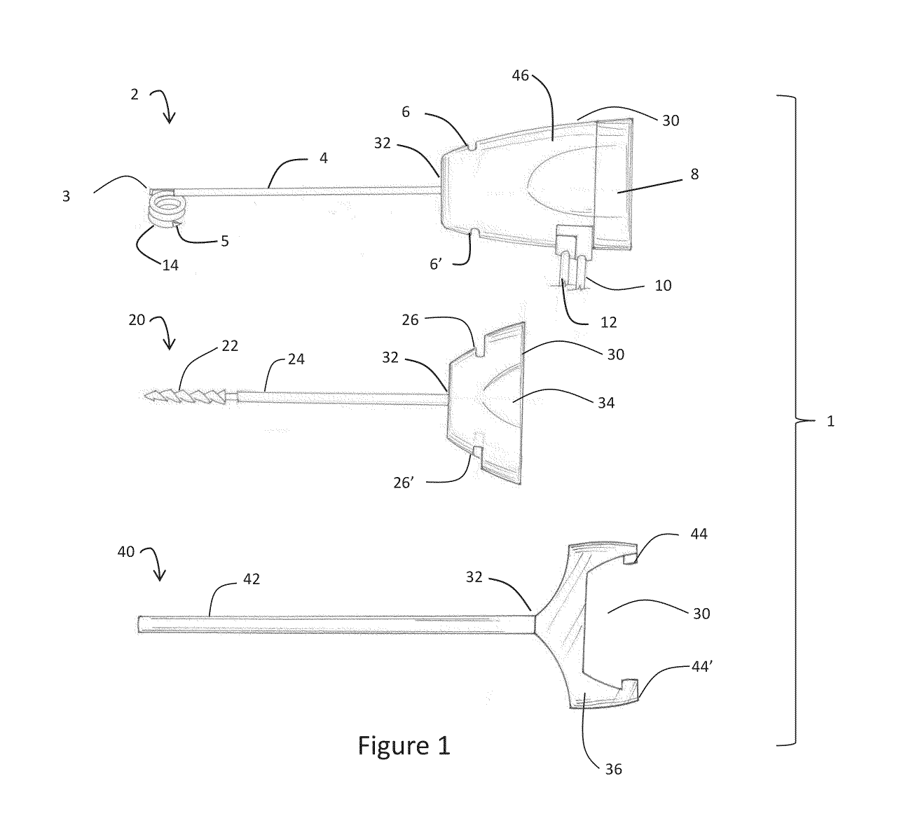

Referring now to FIG. 1, shown therein is an example embodiment of an RF treatment apparatus 1 for providing RF treatment. The RF treatment apparatus 1 generally comprises an RF applicator 2, a guidance hole device 20 and a guidance sheath 40.

The RF applicator 2 comprises an applicator housing 46, an applicator cannula 4 mounted at a distal end 32 of the applicator housing 46, and a handle 8 mounted at a proximal end 30 of the applicator housing 46. An electrode is deployable from the distal end 32 of the applicator cannula 4. The handle 8 acts as an actuator that is used to deploy the electrode. For example, the handle 8 may be a rotary actuator, such as a rotary dial for example, that can be turned manually or assisted by other means depending on the application.

In this example embodiment, the electrode is a coil electrode 14 but in other embodiments, there may be applications in which it may be possible to use other types of electrodes. The RF applicator 2 also comprises locks 6, 6′, such as female finger locks for example, shown spaced from the distal end 32, on a top and a bottom surface of the applicator housing 46. The RF applicator 2 also comprises a tube lead housing 10 and an RF applicator connector 12 connected to the bottom of the applicator housing 46. The RF applicator connector 12 delivers an excitation signal which is used to generate RF energy in use. The tube lead housing 10 slidingly receives the lead end of the coil electrode 14, and houses the electrode 14 when it is in the retracted state.

In at least some embodiments, a slot or groove 3 may be cut out of the distal end 32 of the applicator cannula 4. The slot 3 forces the coil electrode 14 to deploy in a predictable manner and direction. However, there can be embodiments in which the slot 3 is not used.

In this example embodiment, the coil electrode 14 has a helical shape with a tip 5 and the coil electrode 14 may have any variety of cross-sectional shapes including, but not limited to, triangular, circular, semi-circular, squircle or square.

At least the helical portion of the coil electrode 14 can be constructed from a shape memory, electrically conductive alloy to allow for the percutaneous deployment of the coil electrode 14 into a tumor tissue in a minimally invasive fashion. The applicator cannula 4 may be fabricated out of various materials and composites. However, the applicator cannula 4 and the tube lead housing 10 are generally coated, covered or fabricated using an electrically insulated material to provide electrical insulation during use. An example of an electrically insulating material that may be used is clear flexible ⅛″ polyolefin heat-shrink tubing (e.g. ⅛″ Polyolefin Heat-Shrink McMaster Carr, Cleveland, Ohio, US).

The guidance hole device 20 comprises a guidance hole device housing 34 and a guidance hole device cannula 24 mounted at a distal end 32 of the guidance hole device housing 34. The guidance hole device 20 also comprises locks 26, 26′, such as female finger locks for example, spaced from the proximal end 30, on the top and bottom surface of the guidance hole device housing 46. A guidance hole device head 22 may comprise a bone drill, as shown in FIG. 1, a trocar, a bone biopsy needle, a “Murphy” access needle, or any other device that can be used to create a hole in bone tissue such as, but not limited to, cortical and trabecular bone tissue, for example.

The guidance sheath 40 comprises a guidance sheath housing 36 and a guidance sheath sleeve 42 mounted at a distal end 32 of the guidance sheath housing 36. The guidance sheath 40 also comprises locks 44, 44′, such as male finger locks for example, near the proximal end 30, on the top and bottom surfaces of the guidance sheath housing 36 that may be used to engage the locks 6, 6′ or 26, 26′ during use. The guidance sheath sleeve 42 can be sized and shaped to slidingly receive the applicator cannula 4 and the guidance hole device cannula 24. The guidance sheath 40 can also be sized and shaped to slidingly receive other implements, including a PMMA cement injector or a fluid delivery device, such as a needle for example. In other embodiments, additional implements can be utilized in conjunction with the guidance sheath 40. The guidance sheath 40 may be fabricated out of electrically insulated material to provide electrical insulation during use and protect surrounding, non-target, tissues from exposure to RF energy.

In use, the coil electrode 14 has a retracted state when housed within the applicator cannula 4 and a deployed state when moved out of the applicator cannula 4, as shown in FIG. 1. In the retracted state, the coil electrode 14 may exert significant bending force on the applicator cannula 4, such that the applicator cannula 4 may need to be fabricated out of sufficiently rigid material to resist this bending force. The actuator 8 is used to deploy or retract the coil electrode 14, by applying a rotatable force that is generally perpendicular to the direction of insertion of the applicator cannula 4.

The female finger locks 6, 6′, and 26, 26′ can slidingly receive the male finger locks 44, 44′ such that either the RF applicator 2 or the guidance hole device 20 may be locked to the guidance sheath 40. In alternative embodiments, the finger locks 6, 6′, and 26, 26′ can be implemented in different ways to releasably couple one device to another device. For example, in alternative embodiments, the finger locks 6, 6′ and 26, 26′ can instead be meshing threads, snaps, magnets or other suitable elements/structures.

In use, the RF applicator 2 may be slidingly received and locked to the guidance sheath 40. The guidance sheath 40 is held by friction in a guidance hole created by the guidance hole device 20, such that the RF applicator 2 and the guidance sheath 40 are anchored to an element, such as the pedicle for example, as a result of a friction fit on the outside of the guidance sheath sleeve 42 (if a guidance sheath is used). This friction fit, in addition to an optional manual force along the longitudinal axis that can be provided by a user of the RF apparatus 1, holds the RF applicator 2 in place when any resistive forces are encountered due to the deployment of the coil electrode 14. The user may deploy the coil electrode 14 partially with the actuator 8 and then examine the location of the coil electrode 14 with one or more imaging modalities. If the partially deployed coil electrode 14 is correctly placed near the tumor (e.g. target site), the user may continue to advance or retract the coil electrode 14. The coil electrode 14 may be inserted into the tissue at a continuous speed or a modulating speed by using the actuator 8 and a mechanical assembly within the RF applicator 2.

In at least one embodiment, temperature response may be measured and monitored during RF heating. For this purpose, thermal probes (not shown) may be incorporated into the RF applicator 2. For example, optical fibers can be placed along the applicator cannula 4 to measure the thermal distribution of the RF ablation zone and the surrounding tissue during use.

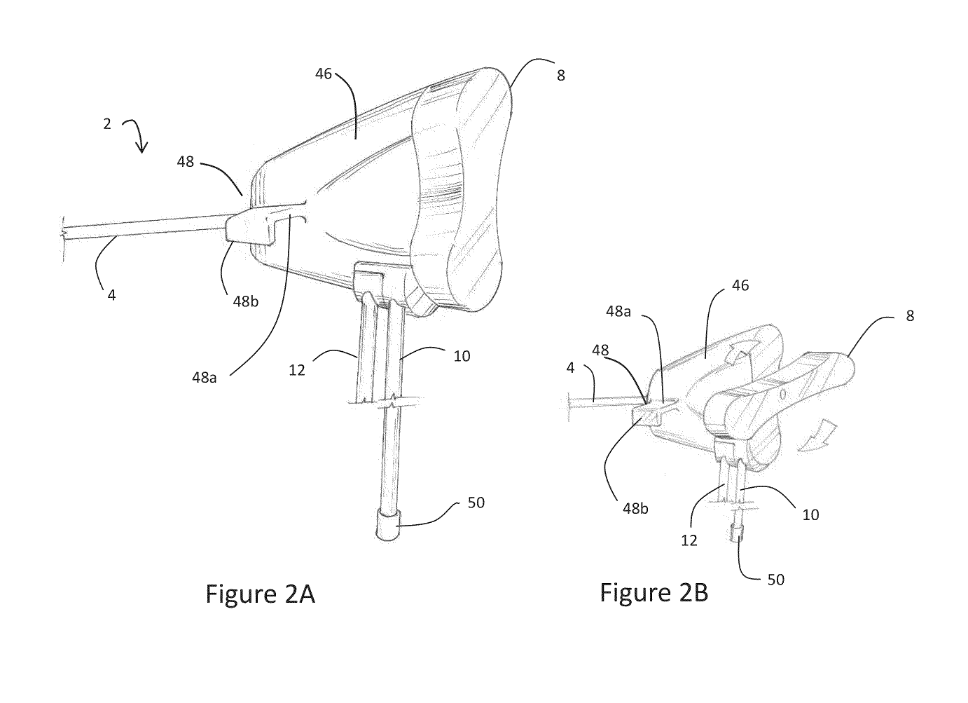

Referring now to FIGS. 2A-2B, shown therein is an example of an alternative embodiment of an RF applicator 2′. In this example embodiment, the direction of the deployment path of the electrode coil 14 is indicated by a visual cue, such as an indicator or indicia, provided on the surface of the RF applicator 2′. An example of such a visual cue is a fin-like guidance protrusion 48. The guidance protrusion 48 comprises a radial fin protrusion 48a that is connected to the applicator housing 46 and may be used to determine the radius or diameter of the helical portion of the coil electrode 14 and the lateral direction of protrusion of the coil electrode 14 when it is moved into its deployed state. The guidance protrusion 48 also comprises an ablation location protrusion 48b that is connected to the radial fin protrusion 48a and may be used to generally determine the center of the RF ablation zone. Also shown in FIG. 2A is the tube lead housing cap 50, which caps the tube lead housing 10.

In FIG. 2B, the actuator 8 is shown in a twisted orientation, which occurs when the actuator 8 is rotated to deploy or retract the coil electrode 14.

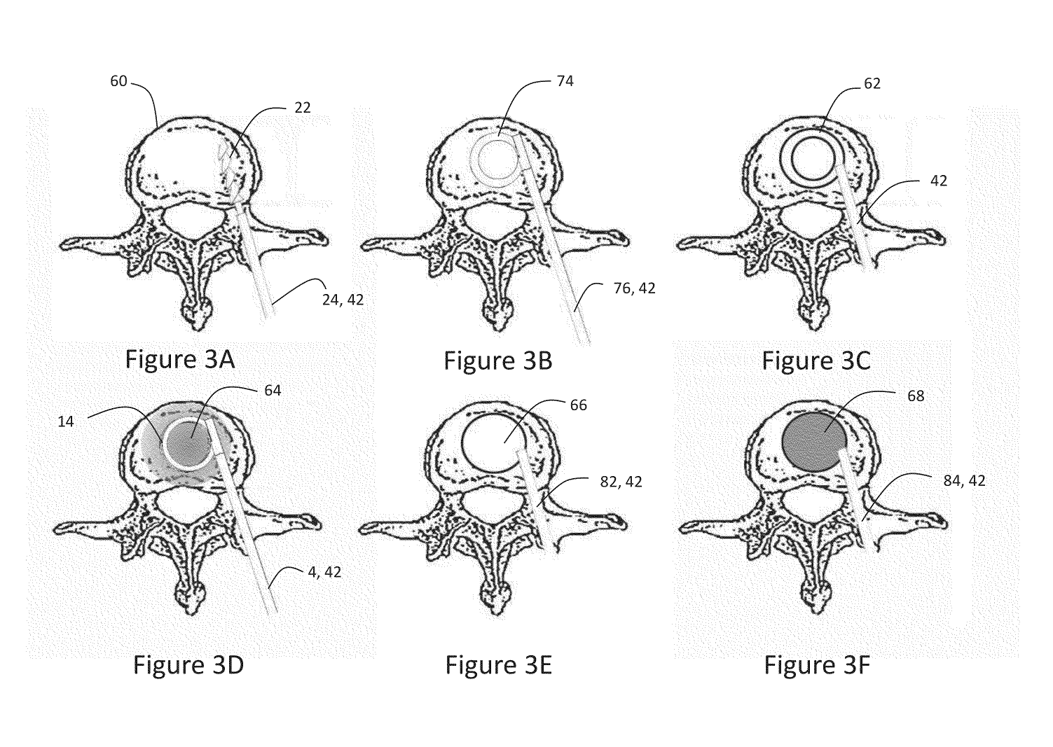

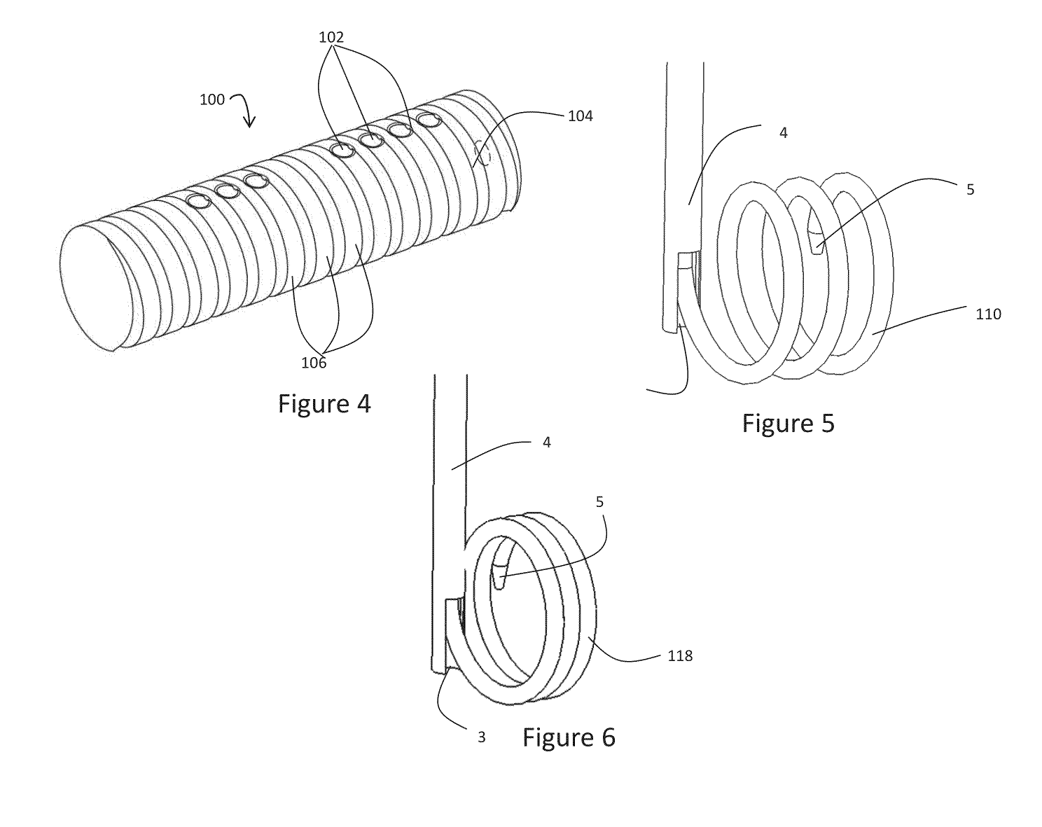

Illustrations that instruct one of an example use of the RF applicator 2 are shown in FIGS. 3A-3F. Although the RF applicator 2 is referred to in this description, other RF applicator embodiments described herein can also be used in a similar fashion as shown in FIGS. 3A-3F.

Referring now to FIG. 3A, according to one example embodiment, with a patient possibly in a lateral decubitus position, the guidance hole device 20 is inserted into a target vertebra using a transpedicular approach to create a guidance hole in the cortical and trabecular bone. In some cases, the guidance hole device 20 may be inserted alone. In other cases, the guidance hole device 20 may be received by the guidance sheath sleeve 42 and then inserted into the patient or subject along with the guidance sheath 40. In some cases, the guidance hole device 20 may also be locked to the guidance sheath 40, such that both devices may be inserted together. For example, the guidance hole device 20 and guidance sheath 40 may be locked or secured to one another using the guidance sheath male finger locks 44, 44′ and the guidance hole device female finger locks 26, 26′. The guidance hole facilitates placement and deployment of the RF applicator 2 in a minimally invasive percutaneous manner. The guidance hole is formed such that the distal tip of the guidance sheath 40 may be located in close proximity to the tumor, such as 2 cm away from the center of the tumor, for example.

The guidance hole device 20 is withdrawn after the guidance hole is made. In the embodiment where the guidance sheath 40 is used, it is unlocked from the guidance hole device 20 and left behind after the guidance hole device 20 is withdrawn.

Referring now to FIG. 3B, the RF applicator 2 is then inserted with the coil electrode 14 in the retracted position. If the guidance sheath 40 was left behind, the RF applicator 2 may be locked to the guidance sheath 40 using the applicator female finger locks 6, 6′, coupled to the guidance sheath male finger locks 44, 44′. In alternative embodiments, the RF applicator 2 may be secured to the guidance sheath 40 using another mechanism, such as, but not limited to, a luer-lock, for example. The coil electrode 14 is then deployed with mechanical assistance via the actuator 8. RF ablation may be carried out during deployment of the coil electrode 14 to facilitate deployment.

In an alternative embodiment, the guidance hole device 20 may be received in a narrow sheath. The guidance hole device 20 is then withdrawn after making the guidance hole and a dilator sheath may be inserted over the narrow sheath. The dilator sheath may be used to widen the original guidance hole and serve as the guidance sheath for the rest of the procedure. The RF applicator 2 or other implements (such as PMMA cement injector or fluid delivery device) may be locked to the dilator sheath while the dilator sheath is inserted, in the same manner that these implements may be attached to the guidance sheath 40, as previously described.

In another alternative embodiment, the RF applicator 2 may be inserted into a secondary guidance sheath which may be inserted with a removable trocar to pierce through the skin of the patient or subject. This secondary guidance sheath may provide the necessary electrical insulation.

Referring now to FIG. 3C, the coil electrode 14 may be deployed out of the tip of the cannula 4 and its placement confirmed by using various imaging modalities such as, but not limited to, Ultrasound (US), Computed Tomography (CT) and Contrast Threshold Imaging (CTF) for example, to position the RF applicator 2 near the tumor. In an alternative embodiment, the RF applicator 2 may have built-in radio-opaque markers to better infer the axial rotation of the RF applicator 2′ in reference to the imaging planes of the selected imaging modality.

Referring to FIGS. 3B-3C, according to an alternative embodiment, a two-tier sequential step may be used when creating the guidance hole. The two-tier sequential step comprises a first step of deploying a first coil 74 in order to create a void 62 that is formed such that it has a shape to allow it to receive a coil electrode. In a second step, the first coil 74 is retracted and a second coil 14 is inserted to encircle the void 62 with at least one full turn. For example, in some cases, the second coil 14 may encircle the void 62 at least 3-4 times in order for optimal RF ablation to occur. The first coil 74 typically has a larger diameter than the second coil 14 and may also be stronger in at least some embodiments. The first coil 74 may also be electrically conductive in some embodiments. RF ablation may be carried out during deployment of the first coil 74, the second coil 14 or both the first coil 74 and the second coil 14. This embodiment may be used when hard trabecular bone is present at the target site, such that resistive forces due to the bone tissue make it difficult to deploy a coil electrode to more than one full turn.

In an alternative embodiment, a conductive substrate may be introduced into the void 62 to ensure proper electrical connectivity. This conductive substrate could be introduced by a fluid delivery system which could be slidingly received within the guidance sheath 40 or could be housed in a lumen of the applicator cannula 4.

Referring now to FIG. 3D, after electrode deployment, the RF applicator 2 is connected to an RF generator (if it is not already connected). For example, the proximal end of the RF applicator 2′ can be connected to the RF generator via a flexible, coaxial cable and matching circuitry. Two or more grounding pads are attached to the bilateral lower back and thigh regions of the patient to complete the electrical circuit required for RF heating. The RF generator is then generally operated within the frequency range of 5-50 MHz at generally a net input power of 50-200 W for a set treatment time in order to ablate or otherwise treat the tumor. The RF generator may output a modulating signal or a constant waveform as the excitation signal. After RF treatment the RF applicator 2 is removed.

In an alternative embodiment, the RF applicator 2 can also be configured in a bipolar arrangement, whereby another coil electrode could be included in the RF applicator 2 such that the grounding pads need not be used.

In another embodiment, sensory feedback may be used to determine the end of the treatment, instead of relying on a set treatment time. The sensory feedback may, for example, include temperature readings from optionally included thermal sensors or reflected power readings measured by the RF generator.

Referring now to FIGS. 3D-3F, the RF treatment technique may comprise a minimally invasive 3-tiered multi-modality regimen. After RF treatment with the coil electrode 14, the target volume may undergo a tissue debulking procedure in which ablated vertebral tissue is removed using a debulking apparatus 82. A hydrocission device is an example of a debulking apparatus. A created void 66 replaces the previous resident tumor tissue and allows for subsequent injection of bone cement into the vertebral body to avoid tumor cell extravasation and venos embolization. Polymethylmethacrylate cement (PMMA) 68, with or without chemotherapeutic agents, may be injected into the vertebral body by a PMMA injection apparatus such as the WE Cook Duroject vertebroplasty cement injector (WE Coom Bloomington Indiana), while under image guidance. This regimen may correct biomechanical abnormalities, stabilize the spine and prevent vertebral body collapse.

In an alternative embodiment, the technique may use a minimally invasive 2-tiered multi-modality regimen, which includes RF ablation with the coil electrode 14 and the use of a PMMA injection apparatus to perform vertebroplasty with PMMA cement doped with or without chemotherapeutic agents.

In various embodiments, the helical portion of the coil electrode 14 can be formed of Nitinol (e.g. NDC-Nitinol Devices & Components, Fremont, Calif., USA). Nitinol has an electrical conductivity similar to that of stainless steel, is MR compatible, biocompatible, and has very high corrosion resistance. However, it has been observed that superelastic metals such as Nitinol exhibit an aged stress-strain curve that is time dependent. For this reason, the helical portion of the coil electrode 14 may not be left in the retracted state, housed within the applicator cannula 4, until right before deployment (for example, within 20 to 30 minutes).

Referring now to FIG. 4, shown therein is an example embodiment of a jig 100 that can be used for setting the shape of the helical portion of the coil electrode 14. The jig 100 is generally cylindrical and comprises helical grooves 106 for retaining the wire, and screw holes 102 can be provided adjacent to the grooves 106, aligned along one side of the jig 100. Screws (not shown) can be fastened to the screw holes 102 to maintain the wire in a set position with the respect to the grooves 106 during annealing.

As an example of a coil electrode, a 1.1 mm Nitinol wire may be wound onto an 11 mm diameter cylindrical jig with a 3 mm pitch helical groove cut into the jig to form the helical portion of the coil electrode (see FIG. 4) such that the coil electrode has a desired length (various lengths, diameters and pitches may be used in alternative embodiments). A tube, such as a needle having the appropriate gauge for example, may be slid over the extending wire from the jig to reduce the curvature of the wire before the annealing process begins. The wire/jig assembly is heat-treated in an annealing oven (e.g. TLD Annealing Furnace, Radiation Products Design, Inc. Albertville, Minn., USA) for 12 minutes at an average temperature of 600° C. In alternative embodiments, alternate heating times and temperatures may be used. After heating, the coil/jig assembly is rapidly cooled at room temperature in water, for example. This heat treatment procedure is designed to produce a superelastic coil electrode whereby mechanical deformation of the coil electrode above its transformation temperature causes stress-induced phase transformation from Austenite to Martensite. The stress-induced Martensite is unstable at temperatures above the Austenite finishing temperature so that when the stress is removed the Nitinol will immediately spring back to the Austenite phase and its pre-stressed shape [10], [11]. Treatment that is longer or shorter than 12 minutes can result in a coil electrode that, when deployed into tissue, will exhibit a higher degree of plastic deformation, coil diameter and pitch expansion. For example, the coil electrode may be fabricated of Nitinol SE510 (e.g. NDC-Nitinol Devices & Components, Fremont, Calif., USA) or similar material with equivalent mechanical properties.

Referring now to FIG. 5, shown therein is a portion of an example embodiment of an RF applicator with a loosely wound helical coil 110 and a guidance groove 3 on the cannula 4. The coil electrode 14, as shown in FIG. 5, can have an 11 mm diameter turn, a 3 mm pitch, and a 1-2 cm length and be fabricated from 1.3 mm diameter round wire of SE 510 Nitinol may be deployed through a 12 ga metal tube into trabecular bone. Improved ablation can result from using at least three turns for the helical portion of the coil electrode. Preliminary testing has demonstrated the potential of this RF applicator for the treatment of tumors in vertebral bodies. Furthermore, the Nitonol material that is used results in a coil electrode that, when deployed at high speeds (for example, deployment of 3 to 4 cm long helical portion within 4 to 5 seconds), exhibits a decrease in final coil expansion, as compared to SE508, for example.

Referring now to FIG. 6, shown therein is a portion of another example embodiment of an RF applicator with a tightly wound helical coil 118 and a guidance groove 3 on the cannula 4. A coil electrode with a tightly wound coil geometry may be preferable when treating target sites (e.g. tumors) in hard substances, such as hard trabecular bone.

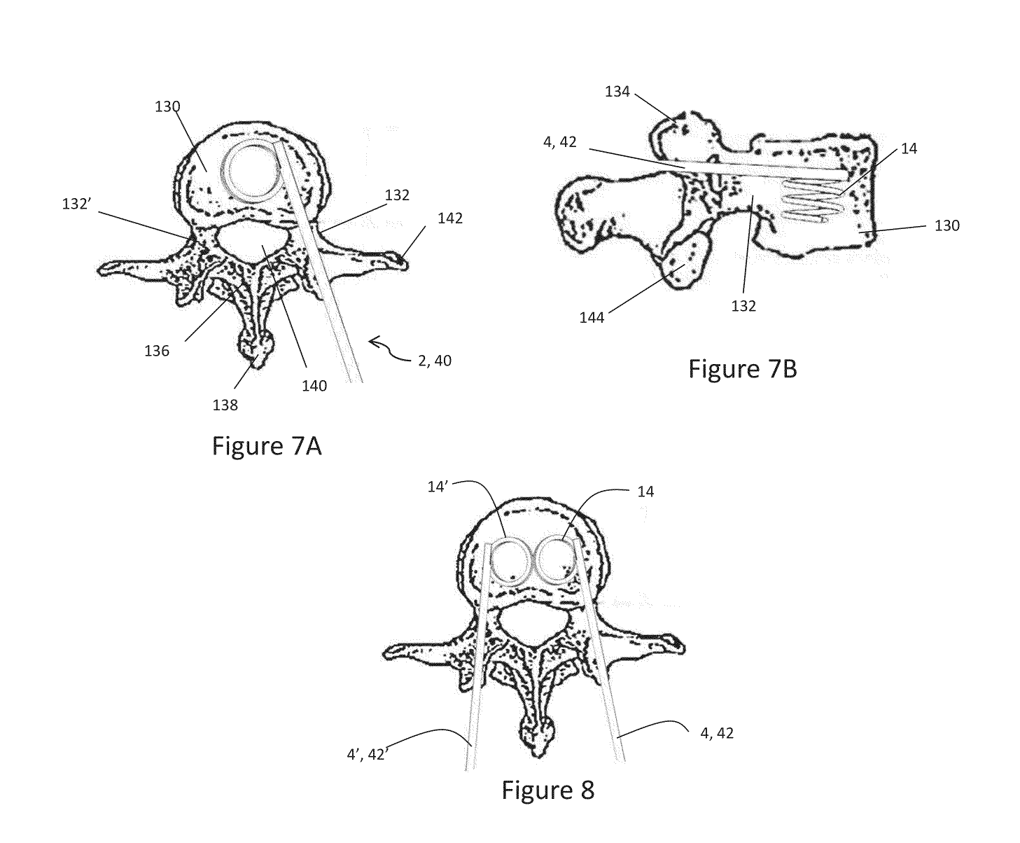

Referring now to FIGS. 7A-7B, shown therein are top and side views, respectively, of an example of a lateral transpedicular coil placement. In particular, the RF applicator 2 and the guidance sheath 40 are shown inserted into a target site in the vertebral body 130 by passing through the pedicle 132 in accordance with the transpedicular approach. The RF applicator 2 is within the guidance sheath 40 with the coil electrode 14 in a deployed state. Also shown are the superior process articular 134, the lamina 136, the spinous process 138, the spinal canal 140, the transverse process 142 and the inferior articular process 144.

Referring now to FIG. 8, shown therein is an example of a bilateral transpedicular coil placement. In this example embodiment, the RF apparatus comprises two RF applicators which may be used to treat multiple target sites or a larger target site within one vertebral body 130. Depending on the patient's diagnosis and tumor size, two coil electrodes 14, 14′ may be deployed simultaneously into the target tissue via a bilateral transpedicular approach, which would enable bilateral transpedicular coil electrode placement. This may be achieved with two separate RF applicators to deploy the independent coil electrodes 14, 14′. The two RF applicators can be connected to a single coax cable or separate coax cables. In an example embodiment, the coil electrodes 14, 14′ may be connected to a single RF system 152.

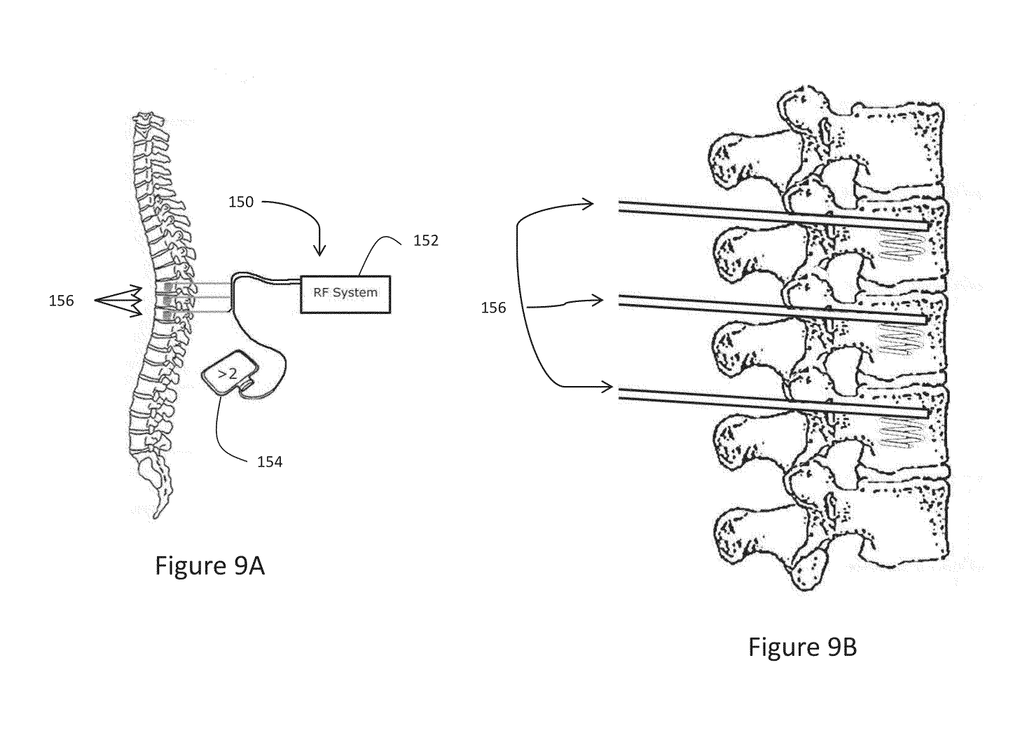

Referring now to FIGS. 9A-9B, shown therein is an example of a multifocal treatment setup. In this case, an RF treatment apparatus can be used that may treat multiple vertebral levels simultaneously such as, but not limited to, anywhere from 2 to 6 vertebrae, for example. In this example, multiple RF applicators 156 may be connected simultaneously to the RF system 152 and at least two grounding pads 154 are positioned on a patient's back while the RF treatment occurs. The RF system 152 may provide an RF excitation signal to each target site simultaneously as a modulating or a constant waveform. In at least one embodiment, the waveforms used for the different treatment sites may be phased with respect to one another. Alternatively, or in addition thereto, the RF system 152 may also treat each site independently through a timed sequenced of delivered RF power. This treatment can be performed in benign or malignant disease tissue.

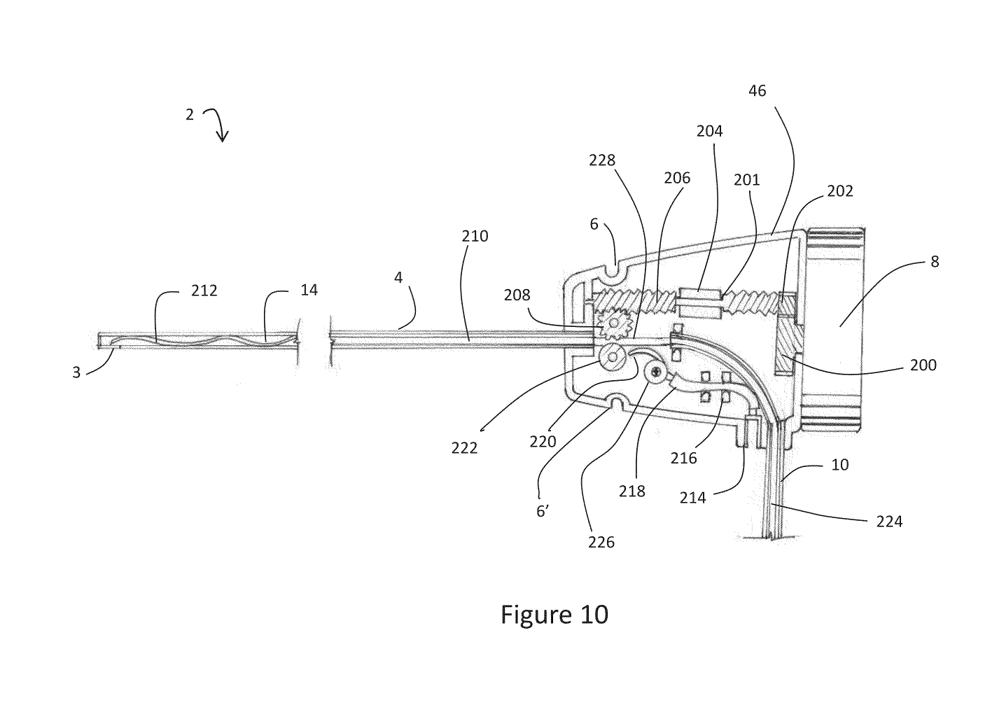

Referring now to FIG. 10, shown therein is a partial cross-sectional view of the RF applicator 2 showing the deployment mechanism that is used to extend and retract the coil electrode 14 during use. The actuator 8 can be configured to move the electrode coil 14 in a manual fashion (not shown) or with assistance. In particular, the actuator and a mechanical assembly within the RF applicator 2 are designed to move a lead portion 210 of the coil electrode 14 in a longitudinal fashion with respect to the applicator cannula 4. The slot 3 ensures that when the lead portion 210 of the coil electrode 14 is moved longitudinally with respect to the applicator cannula 4, the helical portion 212 of the coil electrode 14 is deployed in generally a perpendicular direction with respect to the longitudinal axis of the applicator cannula 4. The mechanical force that is derived from the actuator 8 on the lead portion 210 of the coil electrode 14 is sufficient to overcome any resistive forces due to the friction of the helical portion 212 of the coil electrode 14 on the applicator cannula 4 or any resistance due to deformation from the bone tissue at the deployment site. Manual deployment offers the benefit of tactile feedback and there is less concern regarding sterilization since many of the components used to deploy the coil electrode 14 are disposed within the RF applicator 2. The mechanical assembly employs a suitable amount of leverage which offers the benefit of easier deployment into hard trabecular bone. The mechanical assembly may be implemented in a number of ways other than what is shown. For example, the mechanical assembly may employ a ratchet device in conjunction with the manual manipulation of an actuator, such as a lever or a twist handle; or it may employ a gear train, or it may employ other suitable mechanical parts.

In the example embodiment of FIG. 10, the mechanical assembly comprises a gear train which provides a gear reduction across the mechanical assembly. In this example, the gear train comprises a first gear 200, a worm shaft 201, a second gear 202, a shaft support 204, a worm gear 206, and a worm wheel 208. Other embodiments may use other assemblies of mechanical elements that provide the same functionality as the mechanical assembly shown in FIG. 10. Any of the gears or components that form a part of the RF applicator 2, including the worm wheel 208, may be fabricated from metal or a non-conductive material, such as various types of plastics.

The first gear 200 is disposed near an end of the applicator housing 46 and mechanically coupled to the actuator 8. The worm shaft 201 is disposed along a longitudinal axis of the applicator housing 46. The second gear 202 is coupled to the worm shaft 201 and positioned in order to mesh with the first gear 200. The shaft support 204 is coupled to the applicator housing 46 and to the worm shaft 201 in a manner that prevents the worm shaft 201 from moving in a transverse direction. The worm gear 206 is fixed coaxially with respect to the second gear 202 on the worm shaft 201 and distally with respect to the second gear 202. The worm wheel 208 is disposed within a distal portion of the applicator housing 46 for engaging both the worm gear 206 and the lead portion 210 of the coil electrode 14.

In use, the worm gear 206 retracts or extends the lead portion 210 of the coil electrode 14 depending on the direction of rotation of the actuator 8. In alternative embodiments, the first gear 200 and the second gear 202 may comprise spur gears, helical gears, double helical gears, spiral bevel gears, or any other appropriate gearing device such that actuator 8 is able to drive worm shaft 201. In alternative embodiments, the worm wheel 208 may comprise a spur gear, a helical gear or any other appropriate gearing device such that it is capable of being driven by the worm gear 206 and is able to drive the lead portion of the coil 210.

Given an amount of torque and rotational velocity on the actuator 8, the gear reduction allows additional torque at a lower rotational velocity on the worm wheel 208, and thus lower velocity of deployment of the coil electrode 14. This additional torque may be beneficial for deployment into hard bone tissue. The gear ratios between the first gear 200 and the second gear 202, and between the worm gear 206 and the worm wheel 208, are such that one rotation of the actuator 8 provides less than one rotation of the worm wheel 208, such that a gear reduction exists.

Most of the gear reduction in the gear train occurs between the worm gear 206 and the worm wheel 208, wherein one turn of the worm gear 206 produces only a fractional turn of the worm wheel 208. Furthermore, the use of a worm gear provides the added benefit that any movement of the lead portion 210 of the coil electrode 14, as a result of resistive forces on the coil electrode 14, will not be capable of moving the lead portion 210 of the coil electrode 14 back into the applicator cannula 4. This is because the lead portion 210 of the coil electrode 14 is meshed with the worm wheel 208, which cannot drive the worm gear 206 because of frictional forces.

There may be a gear reduction or a speed multiply between the actuator 8 and the worm shaft 201 depending on the desired deployment speed of the coil electrode 14. In embodiments where a gear reduction is desired, such that one turn of the first gear 200 (that is disposed coaxially with respect to the actuator 8) produces less than one turn of the second gear 202 (that is disposed coaxially with respect to the worm shaft 201), the first gear 200 will be smaller circumferentially and have less gear teeth than the second gear 202. In embodiments where a speed multiply is desired, such that one turn of the first gear 200 produces more than one turn of the second gear 202, the first gear 200 will be larger circumferentially and have more teeth than the second gear 202 (as shown in the example embodiment of FIG. 10).

In at least some embodiments, the lead portion 210 of the coil electrode 14 may be fabricated to mechanically couple with the worm wheel 208. In at least some embodiments, the lead portion 210 of the coil electrode may have cut-outs along its upper surface that are suitably shaped to mesh with the worm wheel 208.

In an alternative embodiment, the mechanical assembly can be configured such that the actuator 8 drives the worm shaft 201 directly without the use of the first gear 200 and the second gear 202. In embodiments where the gears 200 and 202 are included, the first gear 200 and the second gear 202 may be implemented to provide additional gear reduction.

In an alternative embodiment, where additional mechanical reduction is desired between the actuator 8 and the worm shaft 201, an epicyclic gear drive may be included comprising a sun gear disposed at an end of the applicator housing 46 and mechanically coupled to the actuator 8; a plurality of planet carrier gears disposed to mechanically couple with the sun gear; a ring gear fixed to the applicator housing 46 and disposed to mechanically couple with the planet carrier gears; and a joining plate fixed coaxially to the worm shaft and coupled to the planet carrier gears. The joining plate is coupled to the planet carrier gears such that the planet carrier gears may freely rotate about their own individual axis of rotation, but rotation of the plurality of planet carrier gears about the worm shaft axis provides rotation to the joining plate about the worm shaft axis. This coupling may be accomplished by means of pin, roller bearings or any other suitable elements. In use, one turn of the actuator 8 may produce less than one turn of the worm shaft 201. The sun gear, the planetary gear and the ring gear may have spur teeth.

Referring again to FIG. 10, an electrical connection assembly is shown comprising an RF receptacle 214, an electrical cable 218 and an RF brush 220. The electrical cable 218 is coupled to the RF receptacle 212. The RF brush 220 is coupled to the electrical cable 218 and disposed in close proximity to the un-insulated portion 228 of the lead portion 210 of the coil electrode 14 to make an electrical connection during use. In use, the RF applicator connector 12 (see FIG. 1) is inserted into the RF receptacle 214 to deliver an RF excitation signal to the coil electrode 14.

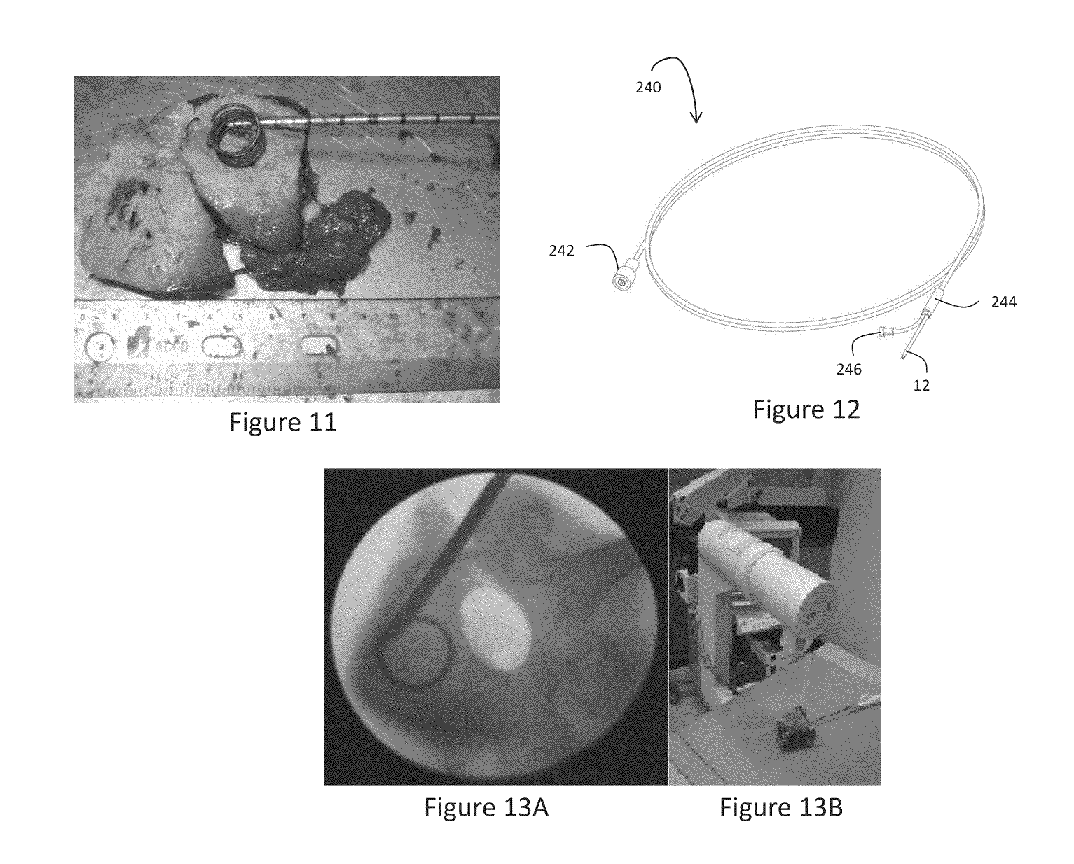

Referring now to FIG. 11, shown therein is a coil electrode that was used in an ex-vivo test in porcine liver tissue. In particular, the RF applicator within a guidance sheath 40 is shown lying on top of a piece of porcine liver tissue after a test.

Referring now to FIG. 12, shown therein is an example embodiment of a cable 240 that can be used with an RF treatment apparatus. The RF delivery cable 240 may be a flexible coaxial cable such as, but not limited to a 6 ft. long RG 58 C/U cable, for example. The RF delivery cable 240 comprises a connector 242, a breakout connection 244 and a grounding electrode connector 246. The breakout connection 244 is proximal to the RF applicator connector 12, which is configured to electrically couple to the RF receptacle 214 (see FIG. 10). The RF delivery cable 240 can comprise an outer metal conductor (not shown) that terminates at a portion of the breakout connection 244 that is coupled to the grounding electrode connector 246. The RF delivery cable 240 can comprise an inner conductor that is connected to the RF applicator connector 12. The grounding electrodes (not shown) may consist of two or more dispersive electrodes (e.g. grounding pads). At the other end, the cable 240 terminates at the connector 242. The breakout connection 244 near the RF applicator connector 12 can limit RF loss along the cable 240. In general, there is no metal shielding between the grounding electrode connector 246 and the RF applicator connector 12, otherwise there may be capacitive coupling which may result in increased impedance and heating of the coaxial cable 240. It has been observed that the RF delivery cable 240 performed adequately when a coaxial cable was used which comprised an inner conducting wire, coaxially surrounded by an insulating material, coaxially surrounded by an outer metal conductor, coaxially surrounded by a braided metal shielding, coaxially surrounded by an insulating coating. The 6 ft. flexible embodiment is user friendly allowing sufficient mobility of the RF applicator 2 around an RF delivery generator and matching circuitry.

Referring now to FIGS. 13A-13B, shown therein is an example coil electrode in excised (i.e. ex vivo) healthy porcine lumbar vertebrae under CT fluoroscopic guidance. In preliminary testing, a coil electrode designed for bone applications (15 mm length, 11 mm diameter, 3 mm pitch, 1.1 mm diameter Nitinol wire, 3 turns) was constructed and tested for deployment under CT Fluoroscopic (CTF) image guidance (see FIG. 13B) in ex vivo models. Later testing showed that a 1.3 mm diameter wire with at least 3 turns and a 3 mm pitch provides better results. Prior to coil electrode deployment, a bone biopsy needle (e.g. SpineJack, Vexim, Saint Jean, France) was used as a guidance hole device to create the guidance hole in the cortical and trabecular bone. This testing showed successful deployment of a single turn of the test coil electrode into the vertebral body (see FIG. 13A). It was found that difficulties may be encountered in complete deployment of all turns of the test coil electrode due to the reactive force in the RF applicator arising from deployment in hard, healthy trabecular bone. By anchoring the RF applicator to a guidance sheath, the RF applicator is effectively secured to the vertebral pedicle due to the friction between the guidance sheath and the pedicle, and thus is able to deliver a greater force to deploy the coil electrode.

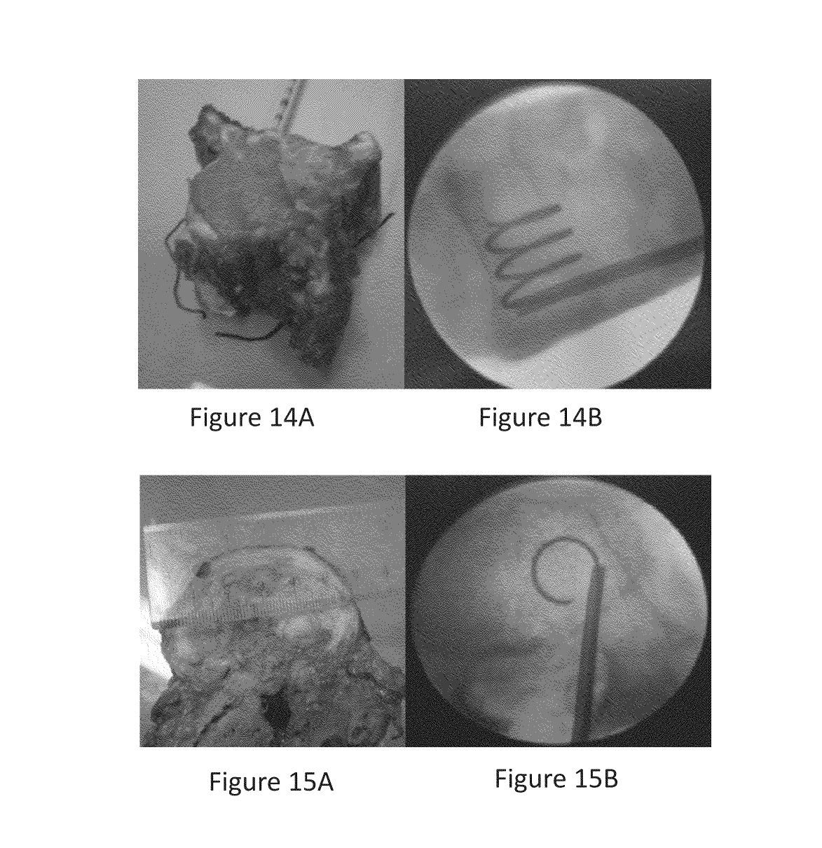

Referring now to FIGS. 14A-14B, shown therein is an example coil electrode in an excised porcine lumbar vertebrae tumor. This was done for further testing in which the coil electrode was deployed into a second model, proposed by [12], in which a cavity was created in porcine lumbar vertebrae and fresh muscle tissue was inserted into the cavity. In preliminary testing, a blind hole measuring 2.5 cm transversely with a 1.5 cm anterior-posterior extent and extending 3 cm inferiorly was created. All turns of the coil electrode were deployed successfully, as shown in FIG. 14B, with no evidence of coil expansion or deformation. A 2 mm margin of clearance was maintained to ensure the coil electrode deployment would not be hindered by the surrounding cortical bone. Another observation from this early investigation demonstrated the importance of coil electrode tip design in determining the initial trajectory of deployment. To follow the desired helical geometry during deployment, it has been found that a guidance hole device with one flattened side at the distal end can help in guiding a wire with a semicircle cross-section to ensure the correct deployment trajectory of the tip. In alternative embodiments, the tip design of the electrode may comprise a beveled tip or a bevel with a tri-facet tip.

Referring now to FIGS. 15A-15B, shown therein is an example coil electrode in excised healthy human cadaveric lumbar vertebrae. Initial coil electrode deployment testing showed successful deployment of a 1¼ turn of a 3 turn coil electrode into the vertebral body. Similar to healthy ex vivo porcine vertebra, difficulties in complete deployment of all turns occurred due to the reactive force in the RF applicator arising from deployment in hard, healthy trabecular bone and the use of a suboptimal guidance sheath. Efforts to secure the guidance sheath to the vertebral pedicle may not support this reactive force. However, the 2-tier sequential step technique discussed above may be used to reduce the reactive force by creating a void for the coil electrode and then deploying the coil electrode.

Further testing evaluated coil electrode geometry in excised bovine liver surrounded by a polyacrylamide bovine serum albumin phantom. The phantom, measuring length 210 mm in length by 160 mm in width by 120 mm in height, was based on an ultrasound phantom recipe developed by [13]. Three grounding pads were casted perpendicular to each other in the phantom located furthest from the coil electrode approximately 16 cm from the middle of the coil perpendicular to a coil electrode axis. An RF generator was operated at 27.12 MHz with net input power value settings of 100 W, 150 W and 200 W (e.g. a Dressler Cesar 273 Power Generator and Matching Network from Advanced Energy, CO, USA). Treatment time was then terminated after the system indicated a severe rapid change in capacitance (>20%) indicating complete ablation. Times ranged from 3 to 4.5 minutes depending on treatment power setting. Temperature was measured inside the centre of the coil electrode using a fluoroptic-based thermometry system (e.g. Luxtron 3100; Luxtron, Calif., USA) with SMM probes. The temperature rapidly elevated with a plateau of 100° C. after a minute with an input power of 100 W. The boundaries of the ablation zones were clearly identified by a denaturation of the structural proteins. The resultant lesion sizes are exhibited in Table 1 below.

TABLE 1
Lesion sizes for various coil electrodes, RF power and time

| | Length | Width | Height | Power | Time | Volume (cm\hat{ }3) |
|---|---|---|---|---|---|---|
| L1 | 35 | 30 | 30 | 200 | 3 | 16.5 |
| L2 | 35 | 35 | 30 | 150 | 3.25 | 19.2 |
| L3 | 30 | 45 | 40 | 100 | 4.5 | 28.3 |

A subsequent study evaluated coil electrode geometry in an established ex vivo tumor model by [12]. In this model, a cavity was created in the vertebral pig body and fresh muscle tissue was inserted into the space. The blind hole was oriented such that it had a 2.5 cm transverse extent, a 1.5 cm anterior-posterior extent and it extended 3 cm inferiorly. All turns of the coil electrode were deployed successfully and a single grounding pad was used. An RF generator was operated at 27.12 MHz with a net input power of 200 W (e.g. a Dressler Cesar 273 Power Generator and Impedance Matching Network from Advanced Energy, CO, USA). The tissue was treated for 10 minutes. On gross inspection, the muscle tissue and surrounding sponge bone was ablated with clear evidence of denaturation of the structural proteins. It was shown that it is feasible to heat and coagulate a large target volume within a short treatment time with the selected coil electrode geometry as described.

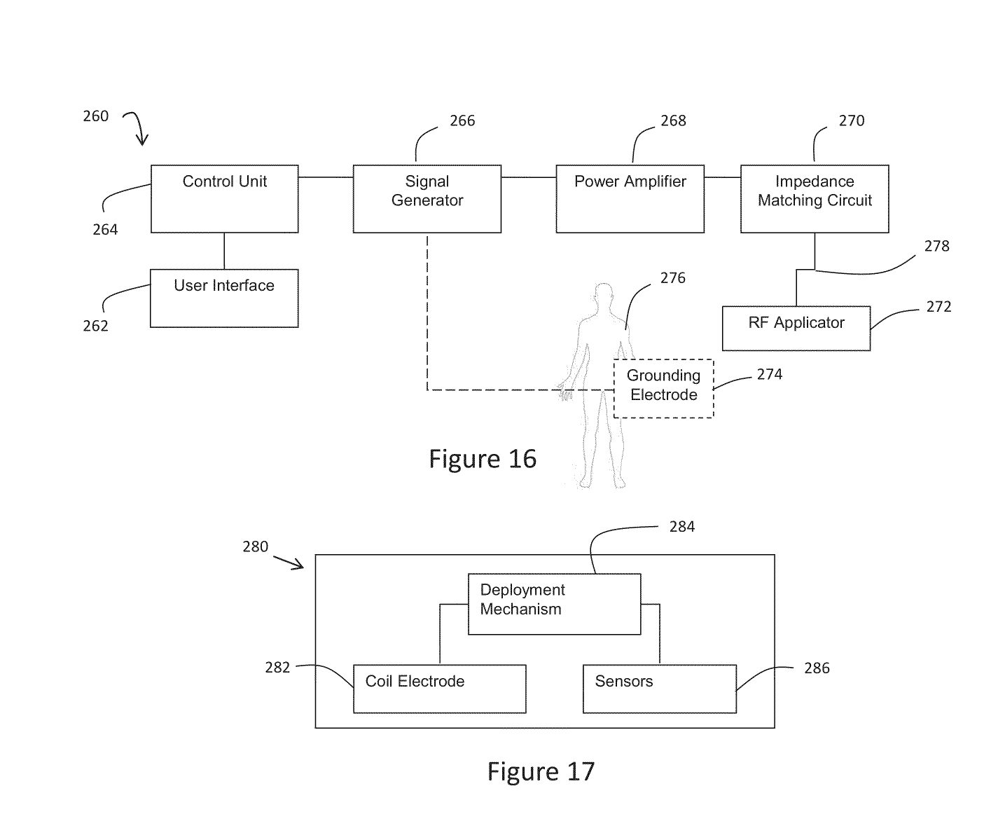

Referring now to FIGS. 16-17, shown therein is a block diagram of an example embodiment of a RF treatment apparatus 260 and a block diagram of an example embodiment of an RF applicator 272, respectively. The RF treatment apparatus 260 includes a user interface 262, a control unit 264, a signal generator 266, a power amplifier 268, an impedance matching circuit 270, and an RF applicator 272. The RF applicator 272 includes a coil electrode 282, a deployment mechanism 284, and one or more sensors 286. Depending on the configuration of the electrode 282, the RF treatment apparatus 260 can additionally (optionally) include a ground electrode 274. The RF treatment apparatus 260, and more particularly the coil electrode 282, and the associated method of operation described herein can be applied to a variety of bone structures.

The RF treatment apparatus 260 can be a standalone device with elements 262-270 provided in a common housing with a cable 278 connecting the impedance matching circuit 270 to the RF applicator 272. In this case, the RF treatment apparatus 260 can also include a power supply along with voltage regulation circuitry (both not shown but are known to those skilled in the art) for providing power to the various components of the RF treatment apparatus 260. Alternatively, the RF treatment apparatus 260 can be configured in a distributed fashion with components 264-270 or subsets thereof being provided by physically separate elements that are connected with cables (in this case cable 278 is still used). In this case, one or more of these components can include their own internal power supply. Furthermore, in this case, the control unit 264 and user interface 262 may not be needed since the components 266, 268 and 270 can each include a user interface for controlling the operation of these components as well as a visual indicator, such as a display or printed labels for associated control dials, to indicate the operational settings of these components.

The control unit 264 controls the operation of the RF treatment apparatus 260 and allows a user, such as a medical practitioner, to heat tumor tissue for a patient, subject or an object (such as a test object) by specifying values for operational parameters using the user interface 262. The patient, subject or object can be a human or an animal or ex-vivo tissue or other material. The control unit 264 can be implemented using a suitable controller or microprocessor as is commonly known by those skilled in the art. The user interface 262 has an input means (not shown) which can include one or more of a keypad, a keyboard, dials, rotary or slide switches, a touch sensitive screen, a mouse and the like that can be used by the user to provide input to the apparatus 260. The user interface 264 can also include a display that can provide feedback to the user, such as graphical or visual display, for example, of the operating parameters of the apparatus 260.

The user can provide input to the RF treatment apparatus 260 for setting its operational parameters. These operational parameters can include the frequency of the excitation current that is generated by the signal generator 266, the power of the excitation current that is applied to the RF applicator 272, the length of time for which the excitation current is applied to the RF applicator 272, the size and location of the tumor, as well as other parameters. The operational parameters can also include safety parameters such as a critical temperature that can be used to disable the operation of the RF treatment apparatus 260 when the temperature of the RF applicator 272 exceeds a certain temperature limit.

The signal generator 266 receives control signals from the control unit 264 to generate an excitation current signal that is applied to the RF applicator 272, after amplification and waveform processing, to generate electric fields that are used to heat the tumor tissue. The signal generator 266 can be considered to be a time-varying current source. The frequency range of the excitation current signal is preferably in the range of 5 to 50 MHz, although this range may be extendable in some cases.

The power amplifier 268 amplifies the excitation current signal to a desired level, which can be on the order of 20 to 500 Watts. For heating tissue in environments with little or no perfusion, power levels as low as 30 to 40 Watts can be effective. A sufficient amount of gain is applied to the excitation current signal so that the large solid tumors are preferably heated to the range of 55 to 90° C. The amount of amplification can be varied depending on the size of the tumor that is to be treated such that the RF applicator 272 can produce a sufficiently large coagulation volume in a single treatment stage. For instance, larger tumors or tumors in highly perfused regions may require a higher heating power from the RF applicator 272. Accordingly, the power amplifier 268 can be a variable gain amplifier.

The impedance matching circuit 270 processes the amplified excitation current signal for maximum power delivery to the RF applicator 272. Accordingly, the impedance matching circuit 270 includes circuitry for matching the impedance of the cable 278, the RF applicator 272, as well as the tissue in which the RF applicator 272 is applied. The length of the cable 278 also has an effect on the impedance matching circuit 270. The configuration of the impedance matching circuit 270 does not have to be appreciably changed when the RF applicator 272 is applied to different types of bone structure. The impedance matching circuit 270 can be implemented using a network of inductors and capacitors, as is commonly known by those skilled in the art. In one example embodiment, the impedance matching circuit 270 includes a capacitor and an inductor connected in series that are used to eliminate the reactive component of the impedance seen downstream from the impedance matching circuit 270, i.e. to achieve resonance. In one example embodiment, a step-down transformer is connected in series with the capacitor and inductor to match the resistive component of the tissue-loaded coil system to the signal generator 266.

In use, the RF applicator 272 is inserted into the tissue of the patient at the site of the tumor. The deployment mechanism 284, an example of which was illustrated in FIG. 10 as the mechanical assembly comprising the gear train in the applicator housing 46 in conjunction with the actuator 8, is used to deploy the coil electrode 282 to surround the target site (e.g. tumor) once the RF applicator 272 has been positioned near the target site. Various imaging modalities can be used to position the RF applicator 272 near the tumor. These imaging modalities include fluoroscopy, magnetic resonance imaging, 3-dimensional cone beam computer tomography and the like. The imaging modalities can also be used to assess therapeutic response following heating of the tumor. The sensors 286 can be optional but if they are used can include temperature sensors, such as a fluoroptic temperature sensor for example, to measure the temperature increase in the tissue during use. In addition, there can be alternative embodiments, which do not include a housing or a deployment mechanism for the coil electrode 282. In cases of very weak bone tissue, such as a Lytic tumor with extensive bone destruction, the RF electrode 282 can be manually slid down a non-conducting channel to the location of the tumor tissue that is to be treated

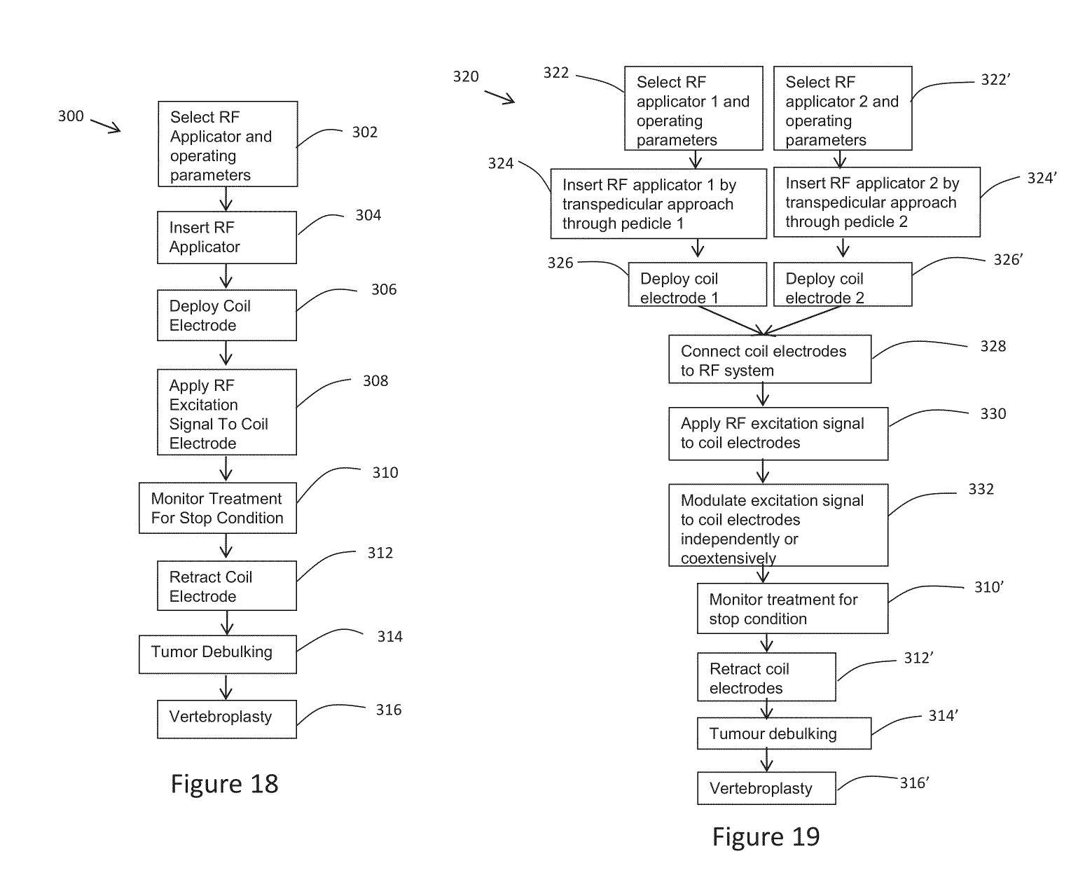

Referring now to FIG. 18, shown therein is a flow chart diagram of an example embodiment of an RF bone tissue treatment method 300. At 302, the parameters of the RF treatment apparatus 260 are selected based on the type and size of the tumor that needs to be treated. The parameters include selecting a particular RF applicator 272 (i.e. size, shape and electrode configuration for the coil electrode 282), an operating frequency, the amount of power of the excitation current that will be applied to the coil electrode 282, and the length of time that the tumor will be exposed to the RF energy provided by the RF applicator 272.

At 304, the RF applicator 272 is inserted into the patient so that the coil electrode 282 can surround the tumor. Image guidance can be used as part of the insertion process. Once the coil electrode 282 is positioned at the target site, the cable 278 can be connected to the impedance matching circuit 20, for example.

At 306, the coil electrode 282 is deployed and if the monopolar configuration is used, then a ground electrode is located on an appropriate location of the patient/subject and connected to the signal generator 266. This may be optional depending on the type of configuration of the coil electrode 282 and the type of RF applicator 272 that is used.

At 308, the excitation signal is applied to the coil electrode 282 with the appropriate amount of power. Based on the geometry of the coil electrode 282 and the operating frequency used, power can be applied continuously for the amount of time required to treat the target tissue. For instance, the target tissue can be heated for about 5 minutes or longer using continuous power provided that it is not too long so as to avoid charring the tissue. The amount of heating can also be determined by measuring tissue temperatures in real-time during application of the RF energy. Either of these conditions can be referred to as a stop condition, which is monitored at 310 to end treatment when the stop condition holds true (i.e. a specified amount of time has elapsed, a specified temperature has been reached, a specified temperature has been measured for a certain amount of time, etc.). A significant increase in reflected power can also be used as a stop condition. However, 310 is optional as the coil electrode 282 can simply be applied to the target tissue to induce heat as needed.

It should be noted that the application of continuous power at 308 is possible based on the geometry of the coil electrode 282 and the operating frequency that is used. With conventional RF ablation technology, electric fields exist mainly at the wire of the electrode, due to the geometry and operating frequencies that are conventionally used. Thermal conduction is relied upon to transfer the generated heat throughout the tumor tissue volume. Accordingly, it takes longer to heat the entire tumor tissue, and the temperature at certain areas of the tissue directly adjacent the wire of the electrode increase in temperature quite a bit which causes the impedance of this tissue area to increase. The increase in tissue impedance reduces the effectiveness of the conventional electrode to heat the tumor tissue. As a result, power to the conventional electrode must be removed, or ramped down, or saline must be inserted at the site, so that the temperature of the heated tissue can decrease, and then the procedure can be applied once more. Accordingly, with conventional electrodes, a “start-and-stop” procedure must be used.

In contrast, the geometry of the coil electrode 282 and the operating frequencies that are used as described herein also produces an axial electric field within the coil electrode 282, i.e. within the volume that is surrounded by the wires of the coil electrode 282, that more effectively and efficiently heats the tumor tissue located within this volume. Accordingly, the RF energy produced by the coil electrode 282 better targets the entire tumor tissue by directly applying generated heat rather than relying on thermal conduction to heat the tumor tissue. Accordingly, in general there are no pronounced increases in temperature, and hence increase in tissue impedance, that require the power to the coil electrode 282 to be removed. Consequently, RF power can be continuously applied to the tumor tissue with the coil electrode 282 until the tumor tissue is fully treated, and the “start-stop” procedure described above for conventional coil electrodes does not have to be used.

After RF ablation at 308 has proceeded until the stop condition is reached at 310, the coil electrode 282 is retracted back into the RF applicator 272 at 312.

Steps 314 and 316 involve a minimally invasive 3-tiered multi-modality regimen. After RF treatment with the coil electrode 282, the target volume may undergo a tissue debulking procedure where ablated vertebral tissue is removed using a debulking apparatus at 314. The created void replaces resident tumor tissue to allow subsequent injection of bone cement into a vertebral body to avoid tumor cell extravasation and venos embolization. At 316, vertebroplasty is performed in which Polymethylmethacrylate cement (PMMA) may be injected into the tumor-affected vertebral body by a PMMA injection apparatus, while under image guidance. This regimen may correct biomechanical abnormalities, stabilize the spine and prevent vertebral body collapse. Steps 314 and 316, either alone or in combination, are optional.

Alternatively, steps 314 and 316 may involve a minimally invasive 2-tiered multi-modality regimen, which includes RF ablation with the coil electrode 282 and using a PMMA injection apparatus to perform vertebroplasty with PMMA cement doped with or without chemotherapeutic agents.

Referring now to FIG. 19, shown therein is a flow chart diagram of an example embodiment of an RF bone tissue bilateral treatment method 320. In this bilateral method, steps 302 to 316 may be performed for two separate coil electrodes in a simultaneous or consecutive fashion. The method 320 relates to the case where the coil electrodes are used together simultaneously. At 322 and 322′, first and second RF applicators and their respective parameters are chosen. At 324, the first RF applicator is inserted through a first pedicle of a vertebra. At 324′, the second RF applicator is inserted through a laterally opposite second pedicle of the same vertebra. At 326 and 326′, the coil electrodes of both RF applicators are deployed. At 328, both RF applicators are electrically connected to a separate or the same RF generator. At 330, an RF excitation signal is applied to both coil electrodes. At 332, this RF excitation signal may be modulated independently or coextensively to the RF electrodes. At 310′, both RF applicators are monitored for a stop condition. At 312′, the coil electrodes are retracted after the stop condition has been met. At 314′, which is optional, tumor debulking is performed. At 316′, which is optional, vertebroplasty is performed.

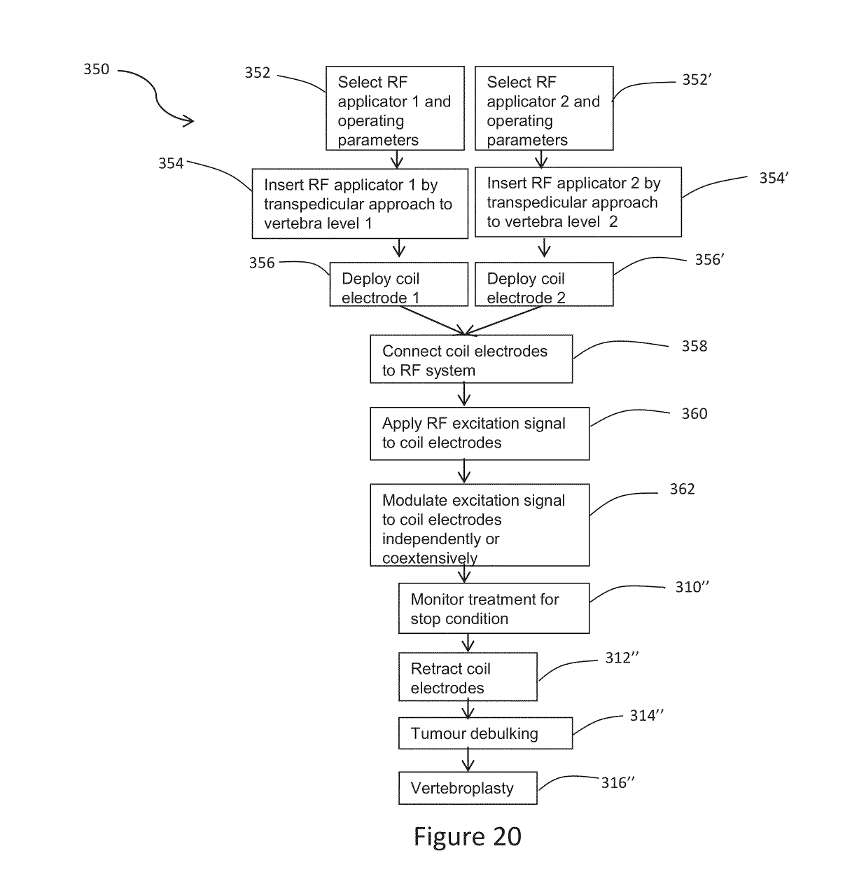

Referring now to FIG. 20, shown therein is a flow chart diagram of an example embodiment of an RF bone tissue multi-modal treatment method 350. In this multi-modal method steps 302 to 316 may be performed for two separate RF applicators in a simultaneous or subsequent fashion. At 352 and 352′, first and second RF applicators and their respective parameters are chosen. At 354, the first RF applicator is inserted through a first pedicle of a first vertebra. At 354′, the second RF applicator is inserted through a second pedicle of a second vertebra. At 356 and 356′ both coil electrodes are deployed. At 358, both RF applicators are electrically connected to the same or separate RF generators. At 360, an RF excitation signal is applied to both coil electrodes. At 360, this RF excitation signal may be modulated independently or coextensively to the RF electrodes. At 310″, the use of the RF applicators is monitored for a stop condition. At 312″, the coil electrodes are retracted after the stop condition is met. At 314″, which is optional, tumor debulking is performed on both vertebrae. At 316″, which is optional, vertebroplasty is performed on both vertebrae. It should be noted that there can be other embodiments of the RF bone tissue multi-modal treatment method 350 in which more than two vertebrae are treated.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments, the general scope of which is defined in the appended claims.

### REFERENCES

[1] Lui, K. W., Gervais, D. A., Arellano, R. A. et al., “Radiofrequency ablation of renal cell carcinoma”, Clinical Radiology, 58: 905, 2003.
[2] Aron, M., Gill, I. S., “Renal tumour ablation”, Current Opinion in Urology, 15: 298, 2005.
[3]. Ahrar, K., Wallace, M. J., Matin, S. F., “Percutaneous radiofrequency ablation: minimally invasive therapy for renal tumours”, Expert Review of Anticancer Therapy, 6: 1735, 2006.
[4] Jemal A. et al., “Annual Report to the nation on the status of cancer, 1975-2005, featuring trends in lung cancer, tobacco use, and tobacco control”, J Nat'l. Cancer Inst., 100(23): 1672-1694, 2008.
[5] Georgy, B. A., “Metastatic Spinal lesions: State-of-the-Art treatment Options and Future Trends”, AJNR. 29:1605-1611, 2008.
[6] Sundaresan N. et al., “Surgery for solitary metastases of the spine: rationale and results of treatment”, Spine, 27: 1802-1806, 2002.
[7] Bilsky, M. H., et al., “Intralesional resection of primary and metastatic sarcoma involving the spine: outcome analysis of 59 patients”, Neurosurgery, 49: 1277-1286, 2001.
[8] Kassamali, R. H., et al., “Pain management in spinal metastases: the role of percutaneous vertebral augmentation”, Annals of Oncology Review, 2010.
[9] Bayer E, Racz G. B., Miles D., Heavner, “Sphenopalatine ganglion pulsed radiofrequency treatment in 30 patients suffering from chronic face and head pain”, Journal of Pain Practice, 5: 223-7, 2005.
[10] Duerig T W, Melton K N, Stöckel D, Wayman C M, Engineering Aspects of Shape Memory Alloys. Butterworth-Heinemann Ltd., Toronto, ©1990.
[11] Prummer, R., Strockel, D., “NITINOL®-Stainless steel compound materials, made by explosive welding”, Fundamental Issues and Applications of Shock-Wave and High-Strina-Rate Phenomena, Proceedings, 581-4, 2001.
[12] Adachi, A., Kaminou, T., Ogawa, T., Kawai, T., Takaki, Y., Sugiura, K., Ohuchi, Y., Hashimoto, M., “Heat distribution in the spinal canal during radiofrequency ablation for vertebral lesions: Study in swine”, Radiology 247(2), 374-80, 2008.
[13] McDonald, M., Lochhead, S., Chopra, R., and Bronskill, M. J., Multi-modality tissue-mimicking phantom for thermal therapy. Physics in Medicine and Biology, 2004, 49, p. 2767-2778.
[14] Kurup et al, J Vasc Intery Radiol, 2010, 21, p. S242-S250.

1. An apparatus for providing RF treatment, the apparatus comprising:
an RF applicator comprising:
an applicator housing;
an applicator cannula mounted at a distal end of the applicator housing, the applicator cannula including a tip spaced apart from a proximal end of the applicator housing;
an electrode disposed within the applicator cannula and the applicator housing, the electrode having a retracted position within the applicator cannula and a deployed position outside of the tip of the applicator cannula;
an actuator mounted at the proximal end of the applicator housing;
a mechanical assembly disposed within the applicator housing to mechanically couple the actuator and the electrode, the mechanical assembly being configured to convert a rotational movement of the actuator to a longitudinal movement of the electrode with respect to the applicator cannula; and
an electrical connection assembly configured to electrically couple the electrode to a power source.

2. The apparatus of claim 1, wherein the electrode is a coil electrode with a helical portion at a distal end of the applicator cannula, wherein the helical portion is within the housing in the retracted position and the helical portion is outside of the tip of the applicator cannula in the deployed position.

3. The apparatus of claim 2, wherein the coil electrode is made of a deformable material.

4. The apparatus of claim 3, wherein the RF applicator comprises a first coil electrode having a first diameter and a second coil electrode having a second diameter that is smaller than the first diameter, wherein the first coil electrode is used to create a toroidal void in bone and the second coil is used to apply RF treatment to a tumor in the bone.

5. The apparatus of claim 2, wherein the coil electrode has a semi-circular cross-section.

6. The apparatus of claim 1, wherein the mechanical assembly includes a gear train comprising:
a first gear disposed at an end of the applicator housing and mechanically coupled to the actuator;
a worm shaft disposed along a longitudinal axis of the applicator housing;
a second gear fixed to the worm shaft and disposed to mechanically couple with the first gear;
a worm gear, fixed coaxially on the worm shaft and disposed distally to the second gear; and
a worm wheel disposed within a distal portion of the applicator housing for engaging both the worm gear and the lead portion of the electrode,
wherein, in use, the worm wheel retracts or extends the lead portion of the electrode depending on the direction of rotation of the actuator.

7. The apparatus of claim 1, wherein the electrical connection assembly comprises:
an RF receptacle;
an electrical cable coupled to the RF receptacle; and
an RF brush coupled to the electrical cable and disposed in close proximity to an un-insulated portion of the coil electrode to make an electrical connection during use.

8. The apparatus of claim 1, wherein the RF applicator further comprises at least one lock located on an exterior surface of the applicator housing, the at least one lock being shaped to couple with at least one lock located on a guidance sheath such that the RF applicator is releasably attachable to the at least one lock of the guidance sheath during use.

9. The apparatus of claim 1, wherein the apparatus further comprises a guidance sheath that is sized and shaped to slidingly receive the applicator cannula, wherein the guidance sheath comprises a guidance sheath housing, a guidance sheath sleeve mounted at a distal end of the guidance sheath housing, and the at least one lock disposed on the guidance sheath housing to releasably attach to the at least one lock on the applicator housing.

10. The apparatus of claim 9, wherein the apparatus further comprises a guidance hole device that is sized and shaped to be slidingly received in the guidance sheath sleeve, wherein the guidance hole device comprises a guidance hole device housing, a guidance hole device cannula mounted at a distal portion of the guidance hole device housing, a guidance hole device head mounted at a distal portion of the guidance hole device cannula and at least one lock located on an exterior surface of the guidance hole device housing, the at least one lock of the guidance hole device being shaped to couple with the at least one lock of the guidance sheath such that the guidance hole device is releasably attachable to the at least one lock of the guidance sheath during use.

11. The apparatus of claim 10, wherein the guidance hole device head comprises one of a bone biopsy needle, a bone drill or a Murphy access device.

12. The apparatus of claim 2, wherein the RF applicator housing further comprises a guidance indicator located on an exterior surface of the applicator housing, the guidance indicator having a first portion mounted to the applicator housing for indicating a direction of deployment and a second portion adjacent the first portion for indicating the center of the electrode when deployed.

13. The apparatus of claim 1, further comprising a slot disposed at a distal end of the applicator cannula, the slot being configured to convert longitudinal movement of the electrode with respect to the applicator cannula to transverse deployment of the electrode with respect to the applicator cannula.

14. A method for providing RF treatment to bone or an intravertebral nerve, wherein the method comprises:
forming a guidance hole adjacent to a target site in the bone;
forming a toroidal void around the target site using a first coil having a first diameter and then retracting the first coil;
deploying a coil electrode having a second diameter in the toroidal void, the second diameter being smaller than the first diameter; and
applying RF energy to the coil electrode to ablate the target site.

15. The method of claim 14, wherein the method comprises deploying the coil electrode until it encircles the toroidal void with at least one full turn.

16. The method of claim 14, wherein the method further comprises introducing a conductive substrate into the toroidal void to improve coupling of the RF energy from the coil electrode to the target site.

17. The method of claim 14, wherein the method further comprises:
ablating the target site to create a void at the target site;
retracting the coil electrode after creating the void at the target site; and
injecting vertebroplasy material into the void at the target site.

18. The method of claim 14, wherein the method further comprises ablation of a sinuvertebral nerve.

19. A method for providing bilateral RF treatment to bone or an intravertebral nerve, wherein the method comprises:
forming a first guidance hole adjacent to a first target site in a vertebrae;
forming a second guidance adjacent to a second target site in the vertebra, the second target site being located laterally opposite the first target site within the vertebra;
inserting and then deploying a first coil electrode at the first target site;
inserting and then deploying a second coil electrode at the second target site; and
applying RF energy to the first and second coil electrodes to ablate the first and second target sites.

20. The method of claim 19, wherein the two coil electrodes are both electrically coupled to a common RF system that provides the RF energy.

21. The method of claim 19, wherein the RF energy is delivered to the first and second coil electrodes independently and coextensively.

22. The method of claim 19, wherein the RF energy is modulated independently or coextensively between the first and second coil electrodes.

23. A method for providing multifocal RF treatment to bone or an intravertebral nerve, wherein the method comprises:
forming a first guidance hole adjacent to a first target site in a first vertebra;
forming a second guidance hole adjacent to a second target site in a second vertebra;
inserting and then deploying a first coil electrode at the first target site;
inserting and then deploying a second coil electrode at the second target site; and
applying an excitation signal to the first and second coil electrodes.

24. The method of claim 23, wherein up to four additional coil electrodes are deployed at a third, fourth, fifth or sixth target sites respectively.

25. The method of claim 23, wherein the coil electrodes are all electrically coupled to a common RF system.

26. The method of claim 23, wherein the RF system is operable to deliver RF energy to the coil electrodes independently and coextensively.

27. The method of claim 23, wherein the RF energy is modulated independently or coextensively between the coil electrodes.

28. The method of claim 23, wherein the method further comprises ablation of a sinuvertebral nerve.
